SEQUENCE LISTING (30 sequences)

SEQ ID NO: 1 - DNA, 14 bp, Artificial
  QPCR Forward Primer Enlu HDC F for the HDCMB21P gene of Enhydra Enhydra lutris
atgttctccgacat

SEQ ID NO: 2 - DNA, 14 bp, Artificial Sequence
  QPCR reverse primer Enlu HDC R for HDCMB21P Gene of Enhydra lutris
gtttcctgcaggtg

SEQ ID NO: 3 - DNA, 14 bp, Artificial Sequence
  QPCR forward primer Enlu COX2 F for COX2 gene of Enhydra lutris
cattcctgatcccc

SEQ ID NO: 4 - DNA, 14 bp, Artificial Sequence
  QPCR reverse primer Enlu COX2 R for COX2 gene of Enhydra lutris
gtccaccccatggc

SEQ ID NO: 5 - DNA, 14 bp, Artificial Sequence
  QPCR forward primer  Enlu CYT F for the Complement cytolysis inhibitor gene of Enhydra lutris
gctggacgagcagt

SEQ ID NO: 6 - DNA, 14 bp, Artificial Sequence
  QPCR reverse primer  Enlu CYT R for the Complement cytolysis inhibitor gene of Enhydra lutris
gacgccagagggag

SEQ ID NO: 7 - DNA, 14 bp, Artificial Sequence
  QPCR forward primer Enlu AHR F for the Aryl hydrocarbon receptor gene of Enhydra lutris
gcgctgagtaccat

SEQ ID NO: 8 - DNA, 14 bp, Artificial Sequence
  QPCR reverse primer Enlu AHR R for the Aryl hydrocarbon receptor gene of Enhydra lutris
cactaagcgtgcat

SEQ ID NO: 9 - DNA, 22 bp, Artificial Sequence
  QPCR forward primer Enlu CYP F for the Cytochrome P450 1A1 gene of Enhydra lutris
ccagtggcagatcgaccatgac

SEQ ID NO: 10 - DNA, 24 bp, Artificial Sequence
  QPCR reverse primer Enlu CYP R for the Cytochrome P450 1A1 gene of Enhydra lutris
ccaaagagaatcaccttctcactc

SEQ ID NO: 11 - DNA, 14 bp, Artificial Sequence
  QPCR forward primer Enlu THR F for the thryroid hormone receptor beta gene of Enhydra lutris
ggacaaaccgaagc

SEQ ID NO: 12 - DNA, 25 bp, Artificial Sequence
  QPCR reverse primer Enlu THR R for the thryroid hormone receptor beta gene of Enhydra lutris
ggaatattgagctaagtccaagtgg

SEQ ID NO: 13 - DNA, 20 bp, Artificial Sequence
  QPCR forward primer Enlu HSP70 F for the heat shock protein 70 gene of Enhydra lutris
ccaggtggcgctgaacccgc

SEQ ID NO: 14 - DNA, 23 bp, Artificial Sequence
  QPCR reverse primer Enlu HSP70 R for the heat shock protein 70 gene of Enhydra lutris
ccttgtagctcacctgcaccttg

SEQ ID NO: 15 - DNA, 23 bp, Artificial Sequence
  QPCR forward primer Enlu IL18  F for the interleukin-18 gene of Enhydra lutris
gtacagaaaacgcatcccatacc

SEQ ID NO: 16 - DNA, 24 bp, Artificial Sequence
  QPCR reverse primer Enlu IL18 R for the heat shock protein 70 gene of Enhydra lutris
ctggaggtctcatttccttaaagg

SEQ ID NO: 17 - DNA, 24 bp, Artificial Sequence
  QPCR forward primer Enlu IL10 F for the interleukin-10 gene of Enhydra lutris
gactttaagggttacctgggttgc

SEQ ID NO: 18 - DNA, 23 bp, Artificial Sequence
  QPCR reverse primer Enlu IL10 R for the interleukin-10 gene of Enhydra lutris
tccacggccttgctcttgttgtc

SEQ ID NO: 19 - DNA, 21 bp, Artificial Sequence
  QPCR forward primer Enlu DRB F for the major histocompatibility complex DRB gene of Enhydra lutris
cggcgagtggagcctatagtg

SEQ ID NO: 20 - DNA, 21 bp, Artificial Sequence
  QPCR reverse primer Enlu DRB R for the major histocompatibility complex DRB gene of Enhydra lutris
ctcctcttcctggccattccg

SEQ ID NO: 21 - DNA, 23 bp, Artificial Sequence
  QPCR forward primer Enlu Met F for the metallothionein gene of Enhydra lutris
gaccccaactgctcctgcgccac

SEQ ID NO: 22 - DNA, 22 bp, Artificial Sequence
  QPCR reverse primer Enlu Met R for the metallothionein gene of Enhydra lutris
gcacagcagctgcacttctctg

SEQ ID NO: 23 - DNA, 18 bp, Artificial Sequence
  QPCR forward primer Enlu CIRBP F for the cold inducible RNA binding protein gene of Enhydra lutris
caaccggtcccgtgggta

SEQ ID NO: 24 - DNA, 17 bp, Artificial Sequence
  QPCR reverse primer Enlu CIRBP R for the cold inducible RNA binding protein gene of Enhydra lutris
cgtagcctcggtcccct

SEQ ID NO: 25 - DNA, 23 bp, Artificial Sequence
  QPCR forward primer Enlu MX1 express F for the MX-1 gene of Enhydra lutris
caagcagctcatcaggaagtaca

SEQ ID NO: 26 - DNA, 18 bp, Artificial Sequence
  QPCR reverse primer Enlu MX1 express R for the MX-1 gene of Enhydra lutris
ggtggcgatgtccacgtt

SEQ ID NO: 27 - DNA, 24 bp, Artificial Sequence
  QPCR forward primer Enlu IL 2 F for the interleukin-2 gene of Enhydra lutris
ggccacagaattgactcatcttca

SEQ ID NO: 28 - DNA, 26 bp, Artificial Sequence
  QPCR reverse primer Enlu IL 2 R for the interleukin-2 gene of Enhydra lutris
cttgtttcagatccctttagtttcag

SEQ ID NO: 29 - DNA, 21 bp, Artificial Sequence
  QPCR forward primer Enlu S9 F for the ribosomal subunit S9 gene of Enhydra lutris
ccagcgccacatcagggtccg

SEQ ID NO: 30 - DNA, 21 bp, Artificial Sequence
  QPCR reverse primer Enlu S9 R for the ribosomal subunit S9 gene of Enhydra lutris
ccctggcctttcttggcgttc

(19) United States
(12) Patent Application Publication
(10) Pub. No.: US 2013/0053269 A1
(43) Pub. Date: Feb. 28, 2013

(54) Primer Sequences for Amplification of Sea Otter Genes, and Methods of Use Thereof

(71) Applicants: A. Keith Miles, Sacramento, CA (US); Lizabeth Bowen, Davis, CA (US)

(72) Inventors: A. Keith Miles, Sacramento, CA (US); Lizabeth Bowen, Davis, CA (US)

(73) Assignee: UNITED STATES DEPARTMENT OF THE INTERIOR, Fort Detrick, MD (US)

(21) Appl. No.: 13/580,015

(22) PCT Filed: Feb. 21, 2011
(86) PCT No.: PCT/US11/25595

§ 371 Date: Sep. 24, 2012

Related U.S. Application Data
(60) Provisional application No. 61/338,966, filed on Feb. 19, 2010.

(51) Int. Cl. C12Q 1/68; C40B 40/06; C40B 30/04; C07H 21/04

(52) U.S. Cl. 506/9; 536/24.33; 435/6.12; 506/16

(57) ABSTRACT

Gene expression technologies have the exciting potential of providing methods for monitoring long-term effects of contaminants and disease on free-ranging marine wildlife species. An added benefit is that these methods may elucidate the mechanisms by which these stressors can deleteriously affect an individual over a long period, and thereby aid in the design of therapeutic and preventative strategies to treat and protect susceptible individuals and populations at risk from oil exposure. Our presentation will assess specific quantifiable genetic markers that can signify persistent pathological and physiological injury associated primarily with chronic hydrocarbon exposure. Using empirical evidence from captive animals and recent captures, we will discuss how we are developing an understanding of gene expression as it relates to the immune system of the sea otter and other marine megafauna, and the potential effects of contaminants or disease.

### CROSS REFERENCE TO RELATED APPLICATIONS

This application claims the benefit of U.S. Provisional Application No. 61/338,966, filed Feb. 19, 2010 which is hereby incorporated by reference herein in its entirety.

### GOVERNMENT INTEREST

This invention was made with support from the United States Government, specifically, the United States Department of the Interior and, accordingly, the United States Government has certain rights in this invention.

### BACKGROUND OF THE INVENTION

1. Field of the Invention

The present invention relates to (1) novel primer sets for amplification of specific sea otter genes, (2) a gene expression assay for measuring pathophysiologic response of sea otters to toxic insults or determining whether toxic insult has occurred, wherein said assay uses these novel primer sets, and (3) methods of use thereof.

2. Background of the Invention

The U.S. Federal Government classifies sea otters (Enhydra lutris) as a threatened species. In the states of California and areas of Alaska, they are classified as threatened as well. Sea otters have been and continue to be subjected to hydrocarbon exposure (i.e., exposure to unrefined crude oil) from a variety of sources, both natural and anthropogenic. An example of significant, direct exposure occurred with the Exxon Valdez oil spill in 1989. The effects of direct exposure to unrefined crude oil are readily apparent. However, the impact of subtle, but pathophysiologically relevant concentrations of crude oil on sea otters and other marine animals is difficult to assess. The challenge is to detect and define the subtle effects of chronic xenobiotic exposure of sea otters.

The acute effects to sea otters of petroleum oil exposure include disturbances in thermoregulation, and lung, liver, kidney damage, and death (Geraci and Williams, 1990; Rebar et al., 1995; Williams et al., 1995). These can and have been detected clinically by hematological and serum chemical analyses or at necropsy. Since the immediate effects of petroleum-based exposure are dramatic, it is relatively straightforward to record, monitor, and study the short-term impacts on sea otters and on populations in the immediate spill area.

A number of studies have documented the long-term impacts of catastrophic oil spills on the marine environment. These may be a result of sub-lethal pathology in individual marine organisms (e.g., birds, mammals, fishes, or invertebrates) exposed to oil at the time of the spill or chronic physiological stresses from continued exposure to oil that lingers in the environment. Whatever the mechanism behind these long-term effects, the pathophysiological changes within an individual organism, though significant, may be subtle and therefore undetectable using classical diagnostic methods. In fact, many of the studies to date investigating low-grade, long-term impacts of oil spills use statistical techniques to identify changes in population demographics, patterns of mortality, reproductive efficiency, and survivability. While the conclusions from these studies are compelling, the supporting data are incomplete and complicated by confounding factors that also impact population demographics and survival.

In marine mammal toxicology, there has been a heavy reliance on the identification and concentration of chemical contaminants within individual tissues or inference from laboratory studies on surrogate species to assess toxic insult from an environmental incident. Unfortunately, these assays are limited in the information they provide since they do not measure how chemical contaminants influence the health of an organism. There is an urgent need to develop sensitive indicators of pathophysiological changes in animals from lingering or low-grade oil-exposure. The animal of particular interest herein is the sea otter.

The present invention addresses these needs, and provides an accurate, non-invasive means for doing so.

### SUMMARY OF THE INVENTION

Gene expression analysis is a tool that can be used to provide the capability to measure an animal's stress response to environmental contamination. The use of gene expression analysis has the potential to transform marine toxicology research (Burczynski, McMillian et al. 2000; Bartosiewicz, Penn et al. 2001). The advantage of using a gene expression assay in marine mammal toxicology lies in its ability to measure the physiologic responses, both acute and chronic, of a marine animals' response to toxic insults. It can be used to detect genes that are activated by particular toxicants, as well as identify subtle changes in an animal's defense responses to toxic insult.

The present invention is based on the use of gene expression analysis to quantitatively measure subtle changes in the sea otter's defense responses to toxic insult. Expression of the genes identified as being up or down-regulated by particular toxicants (i.e., petroleum-based compounds) are useful to monitor the potential exposure of sea otters in the wild to various toxins, and to diagnose and monitor their health due to such exposure.

The invention makes use of genetic information contained in, for example, the blood of sea otters for the diagnosis, prognosis, and monitoring of pathophysiological changes in the sea otter population of a given geographical area. The value of this novel technology is that the up- or down-regulation of many genes, which provide the transcriptional messages important in mediating toxicological and immunological reactions, can be assayed from a single, noninvasive sample of blood.

Microarray analysis and gene-specific quantitative real-time polymerase chain reaction (qRT-PCR) yield important information on the physiological mechanisms that orchestrate an integrated response to a variety of stressors (Marrack, Mitchell et al. 2000). This is an ideal situation for researchers working on wildlife where the amount of sample collected can be limiting.

The invention of the polymerase chain reaction (PCR), a method for amplifying specific sequences and nucleic acids, makes possible the rapid detection of nucleic acids present in a cell in what was previously an undetectably low quantity. Using PCR amplification, one can detect even a single copy of the target nucleic acid. Direct detection by hybridization with the sequence specific oligonucleotide probe of acid sequence amplified to a detectable level makes possible diagnostic tests that are specific enough to detect single nucleotide changes in the sequence. However, not all primer pairs and probes are useful. The choice of primers and, hence, the region to be amplified, along with a choice of probes largely determines the specificity and sensitivity obtainable. The details of PCR are described, for example, in U.S. Pat. No. 4,683,202 (Mullis), U.S. Pat. No. 4,683,195 (Mullis et al.), and U.S. Pat. No. 4,965,188 (Mullis et al.) which are incorporated herein by reference in their entirety. Without going into extensive detail, PCR involves hybridizing primers to the strands of the target nucleic acid (considered “templates”) in the presence of a polymerization agent (such as a DNA polymerase) and be deoxoribonucleoside triphosphates under the appropriate conditions. The result is the formation of primer extension products along the templates, the products having added thereto nucleotides which are complementary to the templates.

Once the primer extension products are denatured, one copy of the templates has been prepared, and the cycle of priming, extending an denaturation can be carried out as many times as desired to provide an exponential increase the amount of nucleic acid which has the same sequence as the target nucleic acid.

In some instances it would be desirable to detect a multiplicity of target nucleic acids (or a multiplicity of nucleic acid sequences in the same nucleic acid) in a single test device. This is referred to herein as “multiplexing”.

Of particular interest is measuring the genomic stress response of sea otters to petroleum-based compounds. Since petroleum-based compounds consist of multiple chemicals or formulations, the toxic effects of exposure and ingestion are likely to be diverse and widespread. For this reason, the utility of a single marker of sub-lethal, oil-induced pathology would be limited. The development of molecular technique(s) capable of detecting toxin-specific patterns in gene expression would permit examination of animals for subtle alterations in multiple physiological processes. Such an approach would facilitate monitoring long-term effects of oil exposure in individual, free-ranging animals such as sea otters.

The present invention provides novel primer sets for use in gene expression analysis to identify and assess stress responses of sea otters to environmental contamination and to monitor sea otters' health. Of particular interest are stress responses of sea otters to subtle petroleum exposure. Each primer set includes a forward primer and a reverse primer. These novel primer sets within the scope of the present invention are set forth in Table 2. Applicants have developed methods for measuring pathophysiological changes in sea otters using these novel primer sets.

Novel primer sets for use in gene expression analysis described herein include:

(1) a primer set including a forward primer having a nucleotide sequence as set forth in SEQ ID NO. 1 (Enlu HDC F), and a reverse primer having a nucleotide sequence as set forth in SEQ ID NO. 2 (Enlu HDC R);

(2) a primer set including a forward primer having a nucleotide sequence as set forth in SEQ ID NO. 3 (Enlu COX2 F), and a reverse primer having a nucleotide sequence as set forth in SEQ ID NO. 4 (Enlu COX2 R);

(3) a primer set including a forward primer having a nucleotide sequence as set forth in SEQ ID NO. 5 (Enlu CYT F), and a reverse primer having a nucleotide sequence as set forth in SEQ ID NO. 6 (Enlu CYT R);

(4) a primer set including a forward primer having a nucleotide sequence as set forth in SEQ ID NO. 7 (Enlu AHR F), and a reverse primer having a nucleotide sequence as set forth in SEQ ID NO. 8 (Enlu AHR R);

(5) a primer set including a forward primer having a nucleotide sequence as set forth in SEQ ID NO. 9 (Enlu CYP F), and a reverse primer having a nucleotide sequence as set forth in SEQ ID NO. 10 (Enlu CYP R);

(6) a primer set including a forward primer having a nucleotide sequence as set forth in SEQ ID NO. 11 (Enlu THR F), and a reverse primer having a nucleotide sequence as set forth in SEQ ID NO. 12 (Enlu THR R);

(7) a primer set including a forward primer having a nucleotide sequence as set forth in SEQ ID NO. 13 (Enlu HSP70 F), and a reverse primer having a nucleotide sequence as set forth in SEQ ID NO. 14 (Enlu HSP70 R);

(8) a primer set including a forward primer having a nucleotide sequence as set forth in SEQ ID NO. 15 (Enlu IL18 F), and a reverse primer having a nucleotide sequence as set forth in SEQ ID NO. 16 (Enlu IL18 R);

(9) a primer set including a forward primer having a nucleotide sequence as set forth in SEQ ID NO. 17 (Enlu IL10 F), and a reverse primer having a nucleotide sequence as set forth in SEQ ID NO. 18 (Enlu IL10 R);

(10) a primer set including a forward primer having a nucleotide sequence as set forth in SEQ ID NO. 19 (Enlu DRB F), and a reverse primer having a nucleotide sequence as set forth in SEQ ID NO. 20 (Enlu DRB R);

(11) a primer set including a forward primer having a nucleotide sequence as set forth in SEQ ID NO. 21 (Enlu Met F), and a reverse primer having a nucleotide sequence as set forth in SEQ ID NO. 22 (Enlu Met R);

(12) a primer set including a forward primer having a nucleotide sequence as set forth in SEQ ID NO. 23 (Enlu CIRBP F), and a reverse primer having a nucleotide sequence as set forth in SEQ ID NO. 24 (Enlu CIRBP R);

(13) a primer set including a forward primer having a nucleotide sequence as set forth in SEQ ID NO. 25 (Enlu MX1 express F), and a reverse primer having a nucleotide sequence as set forth in SEQ ID NO. 26 (Enlu MX1 express R);

(14) a primer set including a forward primer having a nucleotide sequence as set forth in SEQ ID NO. 27 (Enlu IL 2 F), and a reverse primer having a nucleotide sequence as set forth in SEQ ID NO. 28 (Enlu IL 2 R); and

(15) a primer set including a forward primer having a nucleotide sequence as set forth in SEQ ID NO. 29 (Enlu S9 F), and a reverse primer having a nucleotide sequence as set forth in SEQ ID NO. 30 (Enlu S9 R).

An objective of the present invention is to provide a non-invasive method for measuring pathophysiological changes in sea otters using one or more of the novel primer sets herein.

It is an object of the present invention to provide a non-invasive method for detecting exposure of an otter to fuels and other toxins using one or more of the novel primer sets herein.

Yet a further object of the present invention is to provide a kit for measuring through the use of gene expression analysis pathophysiological changes in sea otters using one or more of the novel primer sets herein.

Still a further object of the present invention is to provide a kit for detecting/diagnosing/assessing/monitoring the health of a sea otter, wherein the kit is employed in gene expression analysis. These kits take a variety of forms and compromise one or more probes and, in one embodiment, compromise a panel probes sufficient to determine the identity of the sea otter gene and instructions for using the kit ingredients. The kits can also compromise one or more amplification reagents, e.g., genus specific primers, polymerase, buffers, and nucleic triphosphates.

One aspect of the invention relates to primers for amplifying a specific regions of sea otter nucleic acid. Amplification of the target nucleic acids by PCR, using the primers of the invention, allows one to detect the presence of sea otter nucleic acid by mixing the amplified nucleic acid with the genus specific probes and detecting of hybridization occurs. In a further embodiment, the kit may also comprise positive and negative controls. A preferred positive control is described herein.

Other and further aspects, objects, features, and advantages of the present invention will be apparent from the description of the invention set forth herein.

### BRIEF DESCRIPTION OF THE DRAWINGS

FIG. 1. Is a diagram showing the steps associated with exposure of cell to insult, response of cell to exposure, and eventual production of proteins to combat insult.

FIG. 2 is a graph showing AHR expression in a sea otter, measured by Q-PCR as cycle threshold (Ct)

FIG. 3 is a graph showing MX-1 expression in a sea otter measured by Q-PCR as cycle threshold (Ct).

FIG. 4 is a bar graph showing varying gene expression of two sea otters over time.

FIG. 5 is a bar graph showing average levels of gene expression in 10 genes of 21 sea otters.

FIG. 6 is a bar graph showing levels of gene expression in an adult male sea otter measured by Q-PCR as cycle threshold (Ct).

FIG. 7 is a bar graph showing levels of gene expression in an adult female sea otter measured by Q-PCR as cycle threshold (Ct).

FIG. 8 is a bar graph showing average levels of gene expression in 31 sea otters.

FIG. 9 is a scatter plot graph showing the results of a discriminant funcation analysis comparing levels of gene expression between two populations of sea otters.

FIG. 10 average levels of expression in suite of genes detected in 20 sea otters from Knight Island, 41 sea otters from Montague Island, and 21 sea otters from Katmai, Ak., relative to average values from two healthy otters.

FIG. 11 is a scatter plot graph showing the results of a discriminant funcation analysis comparing levels of gene expression between three populations of sea otters.

FIG. 12 is a box plot graph of the average levels of HDCMB21P gene expression in sea otters from different geographic locations as measured by Q-PCR as cycle threshold (Ct).

FIG. 13 is a box plot graph of the average levels of Aryl Hydrocarbon receptor gene expression in sea otters from different geographic locations as measured by Q-PCR as cycle threshold (Ct).

FIG. 14 is a box plot graph of the average levels of Heat Shock Protein 70 gene expression in sea otters from different geographic locations as measured by Q-PCR as cycle threshold (Ct).

FIG. 15 is a box plot graph of the average levels of Interleukin-2 gene expression in sea otters from different geographic locations as measured by Q-PCR as cycle threshold (Ct).

FIG. 16 is a box plot graph of the average levels of Interleukin-10 gene expression in sea otters from different geographic locations as measured by Q-PCR as cycle threshold (Ct).

FIG. 17 is a scatter plot graph showing the results of a discriminant funcation analysis comparing levels of gene expression between various populations of sea otters.

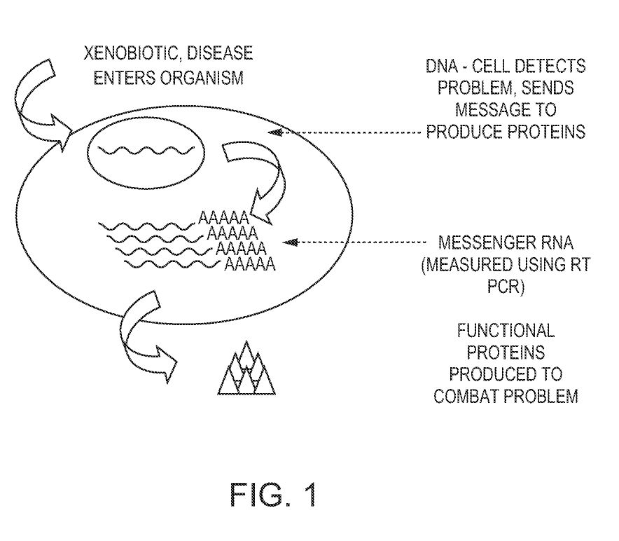

Table 1 provides sea otter genes of interest and their functions.

Table 2 provides novel, sea otter specific QPCR primers.

### DETAILED DESCRIPTION OF THE INVENTION

To aid in understanding the invention, several terms are defined below.

“amplicon” refers to a polynucleotide that is amplified from a bioagent in an amplification reaction.

As used herein, a “bioagent” is any organism, cell, or virus, living or dead, or a nucleic acid derived from such an organism, cell or virus. Examples of bioagents include, but are not limited, to cells, (including but not limited to human clinical samples, bacterial cells and other pathogens), viruses, fungi, protists, parasites, and pathogenicity markers (including but not limited to: pathogenicity islands, antibiotic resistance genes, virulence factors, toxin genes and other bioregulating compounds). Samples may be alive or dead or in a vegetative state (for example, vegetative bacteria or spores) and may be encapsulated or bioengineered.

As used herein, the terms “complementary” or “complementarity” are used in reference to polynucleotides (i.e., a sequence of nucleotides such as an oligonucleotide or a target nucleic acid) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

The term “complement of a nucleic acid sequence” as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids disclosed herein and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. Where a first oligonucleotide is complementary to a region of a target nucleic acid and a second oligonucleotide has complementary to the same region (or a portion of this region) a “region of overlap” exists along the target nucleic acid. The degree of overlap will vary depending upon the extent of the complementarity.

The term “duplex” refers to the state of nucleic acids in which the base portions of the nucleotides on one strand are bound through hydrogen bonding the their complementary bases arrayed on a second strand. The condition of being in a duplex form reflects on the state of the bases of a nucleic acid. By virtue of base pairing, the strands of nucleic acid also generally assume the tertiary structure of a double helix, having a major and a minor groove. The assumption of the helical form is implicit in the act of becoming duplexed.

The term “gene” refers to a DNA sequence that comprises control and coding sequences necessary for the production of an RNA having a non-coding function (e.g., a ribosomal or transfer RNA), a polypeptide or a precursor. The RNA or polypeptide can be encoded by a full length coding sequence or by any portion of the coding sequence so long as the desired activity or function is retained. The terms “homology,” “homologous” and “sequence identity” refer to a degree of identity. There may be partial homology or complete homology. A partially homologous sequence is one that is less than 100% identical to another sequence. Determination of sequence identity is described in the following example: a primer 20 nucleobases in length which is otherwise identical to another 20 nucleobase primer but having two non-identical residues has 18 of 20 identical residues (18/20=0.9 or 90% sequence identity). In another example, a primer 15 nucleobases in length having all residues identical to a 15 nucleobase segment of a primer 20 nucleobases in length would have 15/20=0.75 or 75% sequence identity with the 20 nucleobase primer. As used herein, sequence identity is meant to be properly determined when the query sequence and the subject sequence are both described and aligned in the 5′ to 3′ direction. Sequence alignment algorithms such as BLAST, will return results in two different alignment orientations. In the Plus/Plus orientation, both the query sequence and the subject sequence are aligned in the 5′ to 3′ direction. On the other hand, in the Plus/Minus orientation, the query sequence is in the 5′ to 3′ direction while the subject sequence is in the 3′ to 5′ direction. It should be understood that with respect to the primers disclosed herein, sequence identity is properly determined when the alignment is designated as Plus/Plus. Sequence identity may also encompass alternate or modified nucleobases that perform in a functionally similar manner to the regular nucleobases adenine, thymine, guanine and cytosine with respect to hybridization and primer extension in amplification reactions. In a non-limiting example, if the 5-propynyl pyrimidines propyne C and/or propyne T replace one or more C or T residues in one primer which is otherwise identical to another primer in sequence and length, the two primers will have 100% sequence identity with each other. In another non-limiting example, Inosine (I) may be used as a replacement for G or T and effectively hybridize to C, A or U (uracil). Thus, if inosine replaces one or more C, A or U residues in one primer which is otherwise identical to another primer in sequence and length, the two primers will have 100% sequence identity with each other. Other such modified or universal bases may exist which would perform in a functionally similar manner for hybridization and amplification reactions and will be understood to fall within this definition of sequence identity.

The term “highly conserved,” it is meant that the sequence regions exhibit between about 80-100%, or between about 90-100%, or between about 95-100% identity among all, or at least 70%, at least 80%, at least 90%, at least 95%, or at least 99% of species or strains.

As used herein, the term “hybridization” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, and the Tm of the formed hybrid. “Hybridization” methods involve the annealing of one nucleic acid to another, complementary nucleic acid, i.e., a nucleic acid having a complementary nucleotide sequence. The ability of two polymers of nucleic acid containing complementary sequences to find each other and anneal through base pairing interaction is a well-recognized phenomenon.

The term “oligonucleotide” refers to a molecule comprised of two or usually more the dexoribonucleotides or ribonucleotides, such as primers, probes, nucleic acid fragments to be detected, and nucleic acid controls. The exact size of an oligonucleotide depends on many factors and the ultimate function or use of an oligonucleotide. Oligonucleotides can be prepared by any suitable method, including, for example, cloning and restriction of appropriate sequences and direct chemical synthesis by method known in the art.

The term “primer” refers to an oligonucleotide, whether natural or synthetic, capable of acting as a point of initiation of DNA synthesis under conditions in which synthesis of a primer extension product, complementary to a nucleic acid strand is induced, i.e., in the presence of four different deoxyribonucleic acid triphosphates and an agent for polymerization (i.e., DNA polymerase or reverse transcriptase) in an appropriate buffer at a suitable temperature. A primer is preferably a single stranded oligodeoxyribonucleotide. The appropriate length of the primer depends on the intended use of the primer but typically ranges from 5 to 25 nucleotides. A primer need not reflect exact sequence of the template that must be sufficiently complementary to hybridize with the template and search initiated DNA synthesis. Properties of the primers may include any number of properties related to structure including, but not limited to: nucleobase length which may be contiguous (linked together) or non-contiguous (for example, two or more contiguous segments which are joined by a linker or loop moiety), modified or universal nucleobases (used for specific purposes such as for example, increasing hybridization affinity, preventing non-templated adenylation and modifying molecular mass) percent complementarity to a given target sequences. Properties of the primers also include functional features including, but not limited to, orientation of hybridization (forward or reverse) relative to a nucleic acid template. The coding or sense strand is the strand to which the forward priming primer hybridizes (forward priming orientation) while the reverse priming primer hybridizes to the non-coding or antisense strand (reverse priming orientation). The functional properties of a given primer pair also include the generic template nucleic acid to which the primer pair hybridizes. For example, identification of bioagents can be accomplished at different levels using primers suited to resolution of each individual level of identification. Broad range survey primers are designed with the objective of identifying a bioagent as a member of a particular division (e.g., an order, family, genus or other such grouping of bioagents above the species level of bioagents). In some embodiments, broad range survey intelligent primers are capable of identification of bioagents at the species or sub-species level. Other primers may have the functionality of producing bioagent identifying amplicons for members of a given taxonomic genus, cladespecies, sub-species or genotype (including genetic variants which may include presence of virulence genes or antibiotic resistance genes or mutations). Additional functional properties of primer pairs include the functionality of performing amplification either singly (single primer pair per amplification reaction vessel) or in a multiplex fashion (multiple primer pairs and multiple amplification reactions within a single reaction vessel).

As used herein, the terms “pair of primers,” or “primer pair” are synonymous. A primer pair is used for amplification of a nucleic acid sequence. A pair of primers comprises a forward primer and a reverse primer. The forward primer hybridizes to a sense strand of a target gene sequence to be amplified and primes synthesis of an antisense strand (complementary to the sense strand) using the target sequence as a template. A reverse primer hybridizes to the antisense strand of a target gene sequence to be amplified and primes synthesis of a sense strand (complementary to the antisense strand) using the target sequence as a template. The primers are designed to bind to highly conserved sequence regions of a bioagent identifying amplicon that flank an intervening variable region and yield amplification products which ideally provide enough variability to distinguish each individual bioagent, and which are amenable to molecular mass analysis. In some embodiments, the highly conserved sequence regions exhibit between about 80-100%, or between about 90-100%, or between about 95-100% identity, or between about 99-100% identity. The molecular mass of a given amplification product provides a means of identifying the bioagent from which it was obtained, due to the variability of the variable region. Thus design of the primers requires selection of a variable region with appropriate variability to resolve the identity of a given bioagent. Bioagent identifying amplicons are ideally specific to the identity of the bioagent.

In the disclosed embodiments of the invention, specific sequence primers and probes are provided. It will be apparent to those of skill in the art that provided with those embodiments, specific sequence primers and probes can be modified by, for example, the addition of nucleotides that either the 5′ or 3′ ends, which nucleotides are complementary to the target sequence or are uncomplimentary to the target sequence. So long as primer composition serves a point of initiation for extension on the target sequences, and the primers and probes comprise at least 5 consecutive nucleotides contained within those exemplified embodiments, such compositions are within the scope of the invention.

The term “primer” may refer to more than one primer, particularly in the case when there is some ambiguity in the information regarding one or both ends of the target region to be amplified. If a “conserved” region shows significant levels of polymorphism in a population, mixtures of primers can be prepared that will amplifies such sequences, or the primers can be designed to amplify even mismatch sequences. A primer can be labeled, if desired, by incorporating a label detectable by spectroscopic, photochemical, biochemical, immunochemical, or chemical means. For example, useful labels include 32P, fluorescent dyes, electron-dense reagents, enzymes (as is commonly used in ELISAs), biotin, or haptens and proteins for which antisera or monoclonal antibodies are available. A label can also be used to “capture” the primer, so as to facilitate immobilization of either the primer or a primer extension product, such as amplified DNA, on a solid support.

As used herein, the terms “purified” or “substantially purified” refer to molecules, either nucleic or amino acid sequences, that are removed from their natural environment, isolated or separated, and are at least 60% free, preferably 75% free, and most preferably 90% free from other components with which they are naturally associated. An “isolated polynucleotide” or “isolated oligonucleotide” is therefore a substantially purified polynucleotide.

The term “reverse transcriptase” refers to an enzyme that catalyzes the polymerization of nucleoside triphosphates to form primer extension products that are complementary to a ribonucleic acid template.

The term “sample” in the present specification and claims is used in its broadest sense. On the one hand it is meant to include a specimen or culture (e.g., microbiological cultures). On the other hand, it is meant to include both biological and environmental samples. A sample may include a specimen of synthetic origin. Biological samples may be animal, including human, fluid, solid (e.g., stool) or tissue, as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagamorphs, rodents, otters, minks, sea lions, etc. Environmental samples include environmental material such as surface matter, soil, water, air and industrial samples, as well as samples obtained from food and dairy processing instruments, apparatus, equipment, utensils, disposable and nondisposable items. These examples are not to be construed as limiting the sample types applicable to the methods disclosed herein.

The term “source of target nucleic acid” refers to any sample that contains nucleic acids (RNA or DNA). Particularly preferred sources of target nucleic acids are biological samples including, but not limited to blood, saliva, cerebral spinal fluid, pleural fluid, milk, lymph, sputum and semen. As used herein, the term “sample template” refers to nucleic acid originating from a sample that is analyzed for the presence of “target” (defined below). In contrast, “background template” is used in reference to nucleic acid other than sample template that may or may not be present in a sample. Background template is often a contaminant. It may be the result of carryover, or it may be due to the presence of nucleic acid contaminants sought to be purified away from the sample. For example, nucleic acids from organisms other than those to be detected may be present as background in a test sample.

A “segment” is defined herein as a region of nucleic acid within a target sequence.

A “target” DNA is used in this application also includes nucleic acids which are added to a test specimen to provide positive controls and the assets.

A “PCR reagent” refers to any of the reagents considered essential to PCR, namely primers for the target nucleic acid, a thermostable DNA polymerase, a DNA polymerase cofactor, and one or more deoxyribonucleoside-5′-triphosphates. Other optional reagents and materials used in PCR may be used and are described below.

The term “thermostable polymerase enzyme” refers to an enzyme that is relatively stable to heat and catalyzes polymerization of nucleoside triphosphates to form primer extension products that are complementary to one of the nucleic acid strands of the target sequence.

The present invention may employ conventional molecular biology, microbiology and recombinant DNA techniques within the skill of the art. Some of these techniques are set forth in the literature—i.e., “QIAEX II® Handbook—For DNA extraction from Agarose and polyacrylamide gels and for desalting and concentrating DNA from solutions,” October (2008); “Invitrogen™—TOPO TA Cloning® Kit for Sequencing—Five-minute cloning of Taq polymerase-amplified PCR products for sequencing,” User Manual, Version O (Invitrogen 2006); “PAXgene® Blood RNA Kit Handbook,” Version 2 (PAXgene 2009); Sambook, Molecular Cloning: A Laboratory Manual, Third Edition (Sambook 2001).

Applicants selected fifteen (15) sea otter genes in six different systems as genes of interest. These are set forth in Table 1. These were selected based on Applicants' knowledge of the effects of oil exposure on sea otters. The six different systems from which these genes were selected are (1) immune defense, (2) signal transduction, (3) tumorigenesis, (4) cellular injury, (5) xenobiotic metabolism, and (6) reproduction.

Specific primer sets for these genes were designed, and are set forth in Table 2. The primer sets were designed using the general laboratory methodology, and set forth in greater detail below. Each primer set comprises a forward primer and corresponding reverse primer. These novel primer sets are a result of extensive trial-and-error experimentation. Although Applicants employed conventional, industry-developed techniques in designing these novel primer sets, the specific methodology employed in making these primer sets are a result of considerable experimentation, and required performance of very specific steps under very specific conditions.

The methods of the present invention employ these novel primers for use in identifying and measuring numerous, pathophysiological changes in sea otters due to their exposure to toxins—i.e., oil. These primers may be used to analyze genetic information contained in the blood, or other bodily fluids collected from sea otters so as to assess and monitor the effect of exposure to toxins on the health and wellbeing of the animal. The primers identified herein are “function specific” primers. They amplify genes having specific functions. Note that Table 1 sets forth the function of each gene of interest.

The present invention further comprises a diagnostic or monitoring tool for prognosing or predicting disease or other health issues or, in general, assessing the health of sea otters by detecting the quantitative expression levels of specific genes affected by exposure or possible exposure to toxins, such as petroleum-based compounds. These expression levels are compared to the expressed levels of the same genes obtained from control sea otters that were maintained in clean aquarium environments and routinely monitored for health.

### Development of Sea Otter-Specific Quantitative PCR Primers

Six systems of the sea otter upon which crude oil or components of crude oil were found to have an effect were selected. These six systems include the (1) immune defense, (2) signal transduction, (3) tumorigenesis, (4) cellular injury, (5) xenobiotic metabolism, and (6) reproduction. Fifteen (15) genes within these systems were identified, and are set forth in Table 1.

Degenerate primers for these genes were designed based upon multi-species alignments (GenBank). Once designed, all degenerate or nonspecific primers were ordered from Eurofins MWG Operon (Huntsville, Ala.). Complementary DNA (cDNA) was generated from sea otter blood samples obtained from Dr. Dave Jessup, a California Fish and Game veterinarian permitted by the State and Federal government to maintain captive sea otters. The degenerate primers were utilized on the cDNA generated from the bloods of three sea otters in his facility. Polymerase chain reaction (PCR) amplifications using these degenerate primers were performed on 20 ng of each cDNA sample in 50 μl volumes containing 20-60 pmol of each degenerate primer, 4.0 mM Tris-KOH (pH 8.3), 15 mM KOAc, 3.5 mM Mg(OAc)₂, 3.75 μg/ml bovine serum albumin (BSA), 0.005% Tween-20, 0.005% Nonidet-P40, 200 μM each dNTP, and 5 U of ADVANTAGE™ 2 Taq polymerase (Clontech, Palo Alto, Calif.). The PCR was performed on an MJ Research PTC-200 thermal cycler (MJ Research, Watertown, Mass.), and consists of 1 cycle at 94° C. for 3 minutes, 40 cycles at 94° C. for 30 seconds, 60° C. for 30 seconds, and 72° C. for 2 minutes, with a final extension step of 72° C. for 10 minutes. The products of these reactions were electrophoresed on 1.5% agarose gels and visualized by ethidium bromide staining. Bands representing PCR products of the predicted size were excised from the gel, and extracted and purified using a commercially available nucleic acid-binding resin (QIAEX II™ Gel extraction kit, (QIAGEN, Valencia, Calif.).

Isolated fragments were ligated into a T/A type cloning vector (pGEM-T EASY™ vector systems; Promega, Madison, Wis.). Following transformation, growth, and blue-white selection in competent cells (SE DH5α competent cells, Life Technologies Inc., Rockville, Md.), the DNA from positive clones was isolated. Nucleotide sequences of both strands were determined by dideoxy nucleotide methodology using an automated sequencer (Model 373; Applied Biosystems, Foster City, Calif.). Nucleotide sequences of the PCR products were analyzed using ALIGN™ and CONTIG™ sequence alignment software programs (Vector NTI™; Informax Inc., North Bethesda, Md.) and compared to known sequences using the NCBI BLAST program, and the IMGT/HLA database.

Sea otter-specific quantitative PCR primers within the scope of the present invention were subsequently designed using Primer Express (primer design software, Applied Biosystems, Foster City, Calif.).

### Experimental Procedures and Examples

Sea otters were captured within each of two geographically distinct populations (California and southeast Alaska) using tangle nets, hand-held dip nets, or under-water diver-held traps. Captured animals were anesthetized, weighed, measured, and blood samples obtained. Following sampling, anesthesia was reversed and the animal was released near the capture location. All captures were conducted under existing Federal OMA Permits issued to Mr. J. Bodkin and Dr. M. T. Tinker, United States Department of the Interior, United States Geological Survey (USGS), and procedures approved by the USGS Animal Care and Animal Use Committee according to Federal guidelines.

Blood was immediately injected into PAXgene blood RNA collection tubes (PreAnalytiX, Switzerland), refrigerated in the field, and then frozen at the USGS Western Ecological Research Center facilities until processing. Rapid RNA degradation and induced expression of certain genes after blood draws has led to the development of methodologies for preserving the RNA expression profile immediately after blood is drawn. PAXgene tubes contain a blend of RNA stabilizing reagents that protect RNA molecules from degradation by RNases and prevent induction of gene expression. Without this stabilization, copy numbers of individual mRNA species in whole blood can change more than 1,000-fold during storage and transport.

RNA from blood in the PAXgene tubes was then isolated according to manufacturer's standard protocols (silica-based microspin technology). The extracted RNA was treated with 10 U/μl of RNase free DNase I (DNase, Amersham Pharmacia Biotech Inc., Piscataway, N.J.) to remove contaminating gDNA at 37° C. for 20 minutes followed by heat inactivation at 95° C. for 5 minutes and chilling on ice. Extracted RNA was stored at −80° C. until processing and analysis.

A standard cDNA synthesis was performed on 2 μg of RNA template from each animal. Reaction conditions included 4 units reverse transcriptase (OMNISCRIPT™, Qiagen, Valencia, Calif.), 1 μM random hexamers, 0.5 mM each dNTP, and 10 units RNase inhibitor, in RT buffer (Quiagen, Valencia, Calif.). Reactions were incubated for 60 minutes at 37° C., followed by an enzyme inactivation step of 5 minutes at 93° C. and stored at −20° C. until further analysis.

Quantitate Real-time PCR (Q-RTPCR) systems for sea otter S9 gene (18S ribosomal subunit/housekeeping gene—reference gene that does not change as a result of exposure to toxin) and the genes of interest were run in separate wells using the cDNA synthesized and the primer sets identified in Table 2. There were fifteen separate wells, one for each gene of interest, run in duplicate. Complimentary DNA was examined using an intercalating fluorescent dye PCR. Each reaction contained 500 ng DNA in 25 μl volumes with 20 pmol SSP, Tris-Cl, KCl, (NH₄)₂SO₄, 2.5 mM MgCl₂ (pH 8.7), dNTPs, HOTSTAR™ Taq DNA Polymerase (Quantitect SYBR Green PCR Master Mix, Qiagen, Valencia, Calif.), and 0.5 units uracil-N-glycosylase (Rocke, Indianapolis, Ind.). Amplifications were performed in an ABI 7300 (Applied Biosystems, California) under the following conditions: two minutes at 50° C., followed by 15 minutes at 95° C., and 40 cycles of 94° C. for 30 seconds, 58° C. for 30 seconds, and 72° C. for 30 seconds. Reaction specificity was monitored by melting curve analysis using a final data acquisition phase of 60 cycles at 65° C. for 30 seconds and verified by direct sequencing of randomly selected amplicons.

Gene expression is measured as Ct values—threshold crossing values. The lower the Ct value, the more the gene is expressed. Gene expression data obtained from these samples were then compared to similar data generated from samples obtained from control animals in known and documented good health. These control animal samples were obtained from captive sea otters residing at the Vancouver Aquarium, Vancouver, British Columbia, Canada and Shedd Aquarium, Chicago, Ill. As we increase our sample size from known healthy captive sea otters, we will be able to establish diagnostic ranges of expression that can be used as a standard for field investigations and veterinary use. These ranges would be able to be determined by one having ordinary skill in the art having knowledge of the present invention.

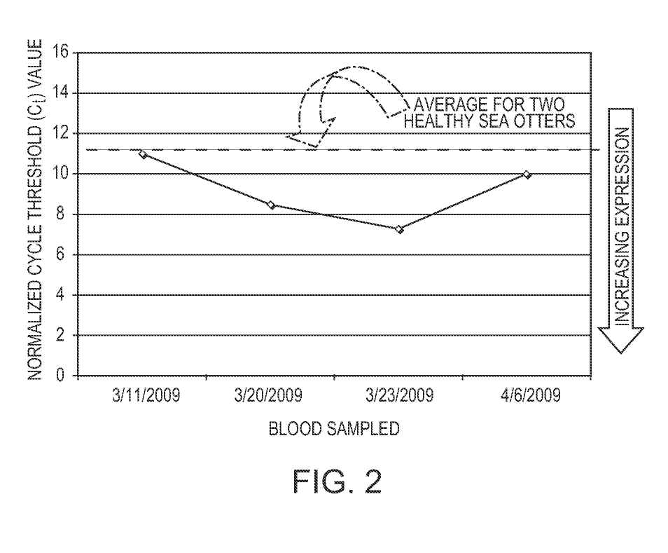

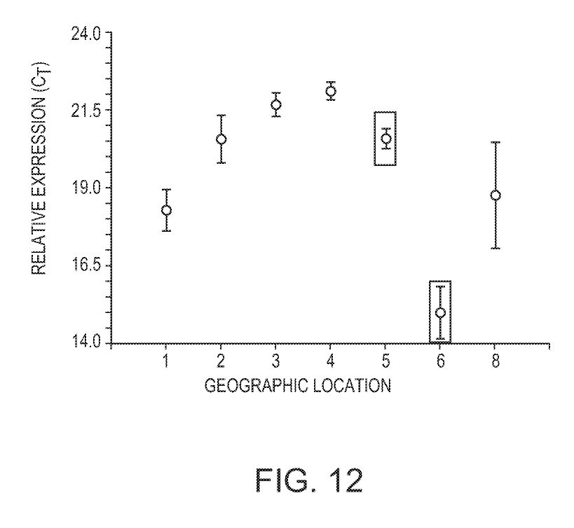

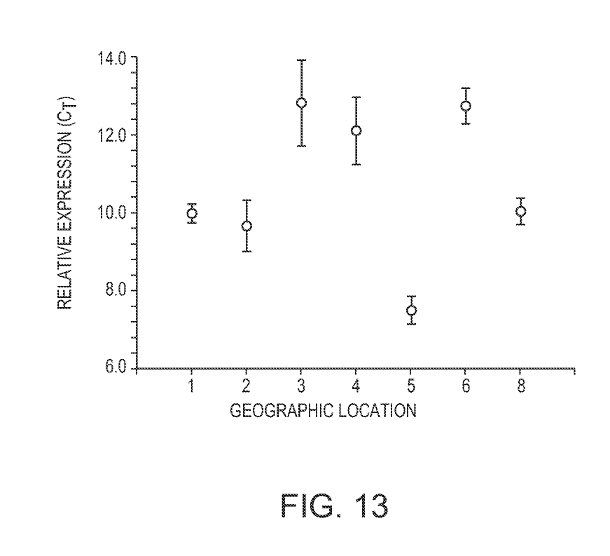

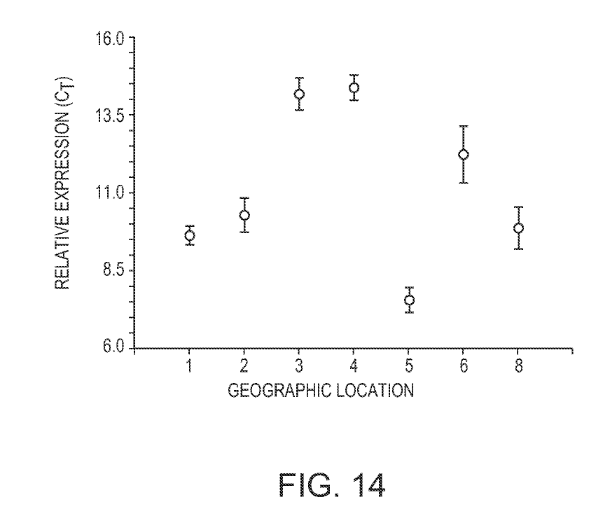

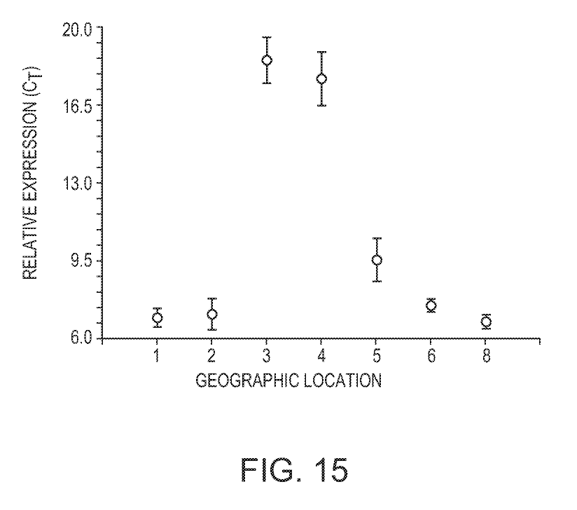

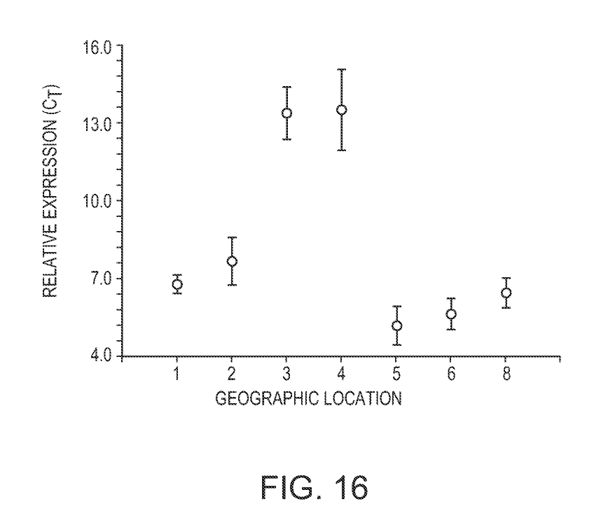

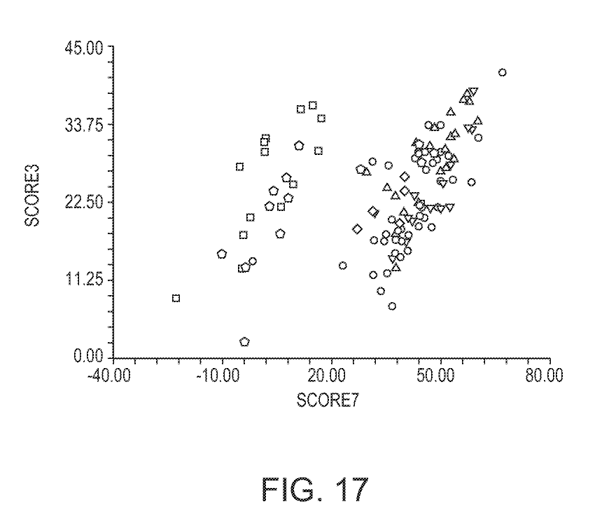

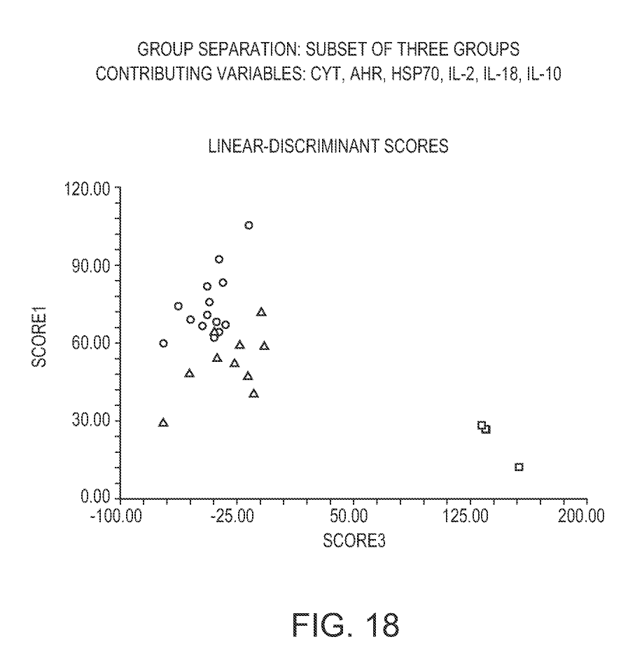

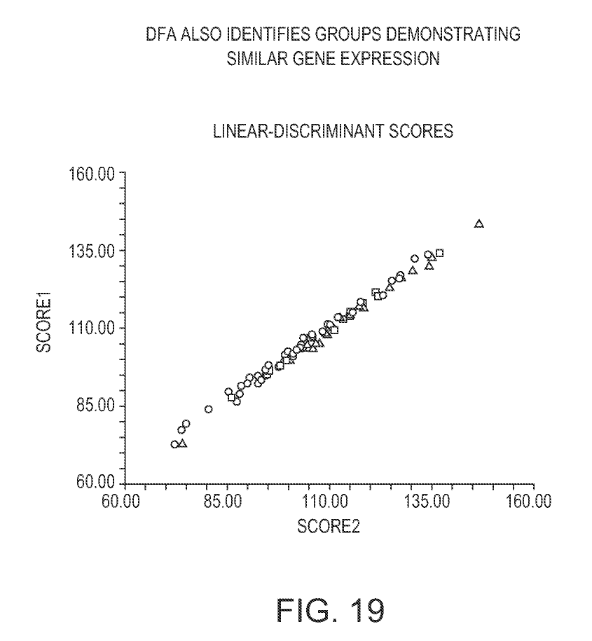

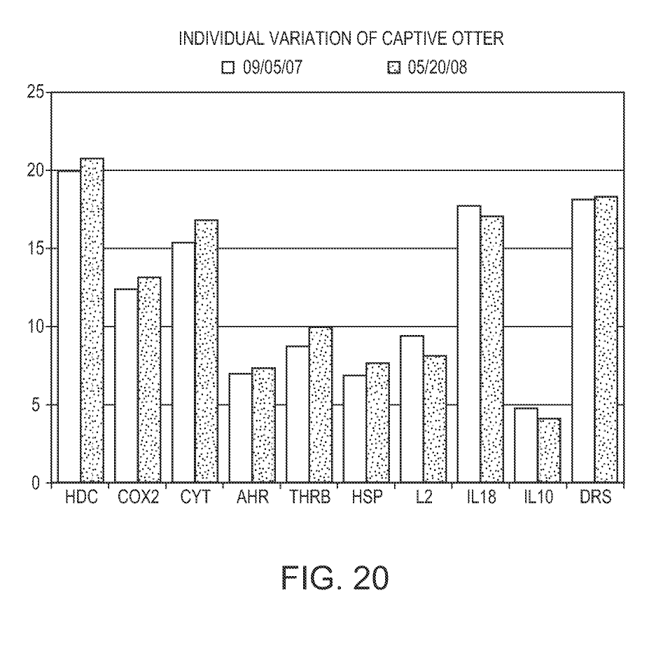

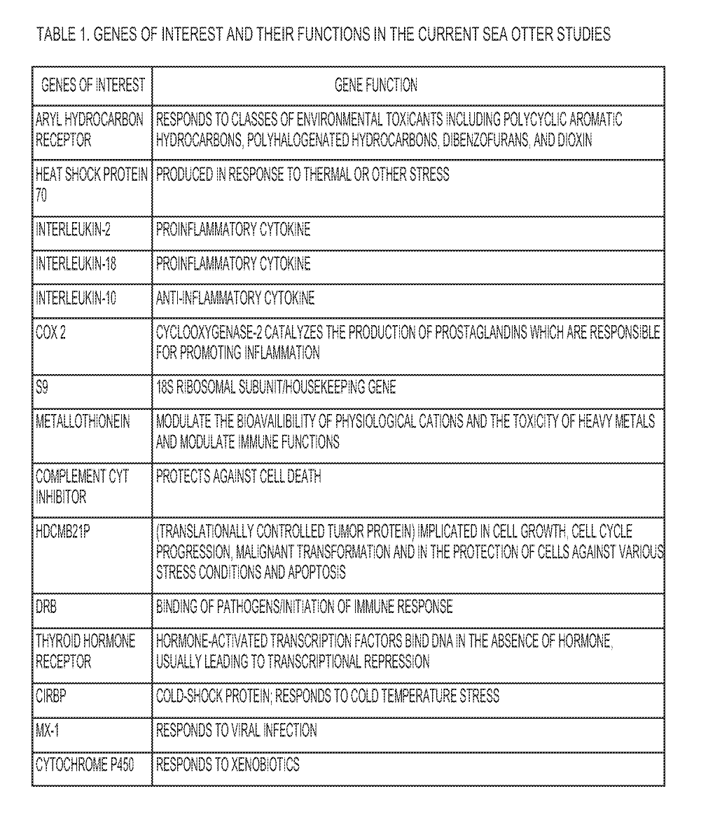

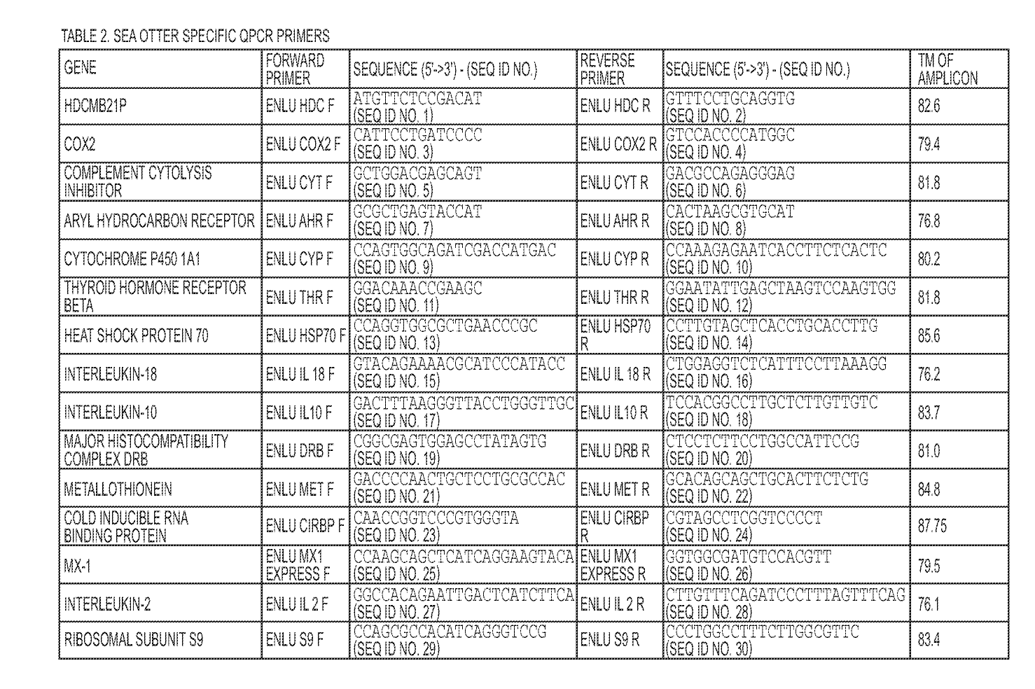

The experimental procedure described above was used in the following Examples (case studies), the results of which are graphically set forth in FIGS. 2-20.

### Example 1

### Case Study: Olive the Oiled Sea Otter

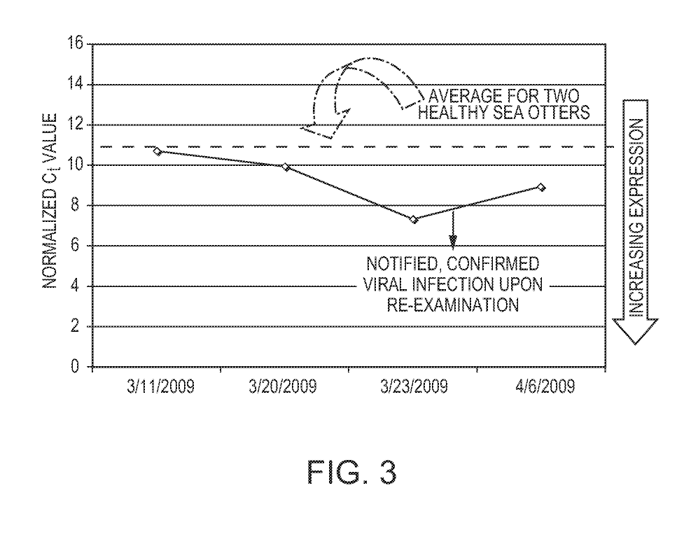

This sea otter suffered exposure to oil from a natural seep, and was captured by California Fish and Game. Blood was drawn at the time of capture and periodically during its rehabilitation at the Monterey Bay Aquarium. Using the novel primers as described herein, an increase in Aryl Hydrocarbon Receptor (AHR) expression was observed, followed by a decrease in expression during rehabilitation. FIG. 2 provides a graphical representation of this with reference to the average AHR expression of two healthy sea otters from the Vancouver Aquarium. In addition, an increase in MX-1 expression was observed after oiling indicating viral infection possibly exacerbated by exposure that decreased with rehabilitative care—see FIG. 3.

### Example 2

### Case Study: Three Sea Otters from Shedd Aquarium

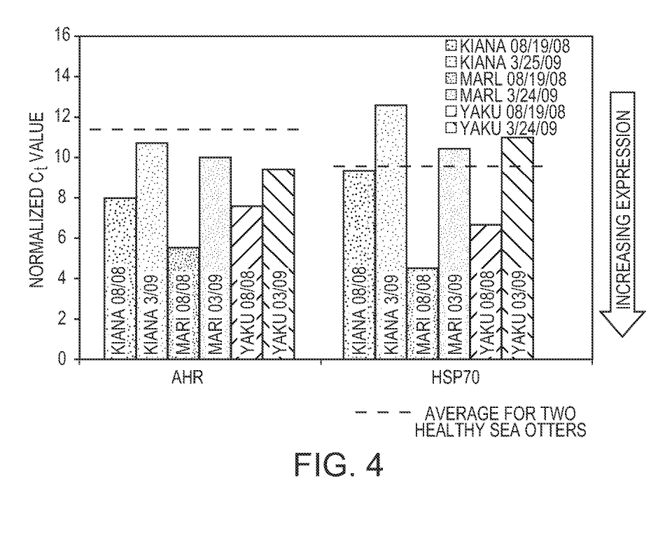

Blood samples were obtained from three Shedd Aquarium sea otters (Chicago, Ill.) before being transported to a temporary facility in Minnesota. The three sea otters are identified as Kiana, Mari and Yaku. Using the novel primers as described herein, a high expression of AHR and HSP70 (heat shock protein 70) was observed. This was probably indicative of less than ideal conditions at the Chicago facility that was in need of and scheduled for renovation. Expression of these genes decreased after they were removed from the Shedd Aquarium—see FIG. 4. Note that the normalized Cₜ values are provided in the following order, from left to right—Kiana Aug. 19, 2008, Kiana Mar. 25, 2009, Mari Aug. 19, 2008, Mari Mar. 24, 2009, Yaku Aug. 19, 2008 and Yaku Mar. 24, 2009, respectively first for expression of AHR, and then for expression of HSP70. Note that FIG. 4 illustrates this with reference to the average corresponding gene expression for two healthy sea otter.

### Example 3

### Case Study: 21 Sea Otters from Katmai, Ak.

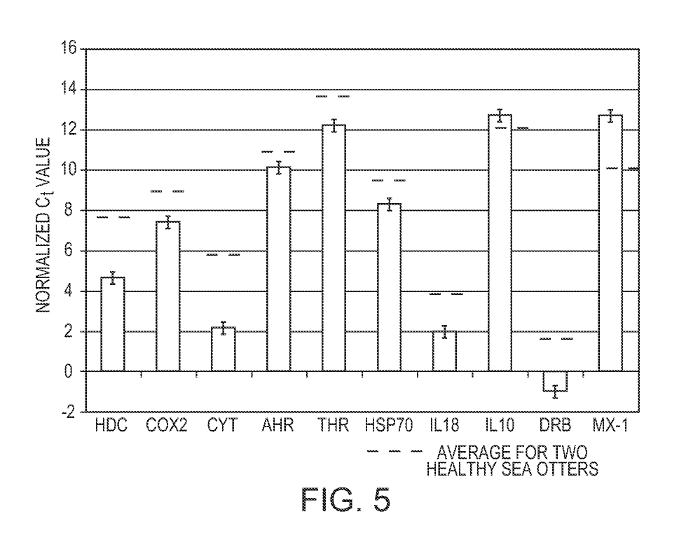

Blood samples were obtained from twenty-one (21) sea otters from Katmai, Ak. Using the novel primers as described herein, the average levels of expression in ten (10) genes was determined, and compared with the average values from two healthy sea otters—see FIG. 5. The Katmai sea otter population was assumed to be relatively healthy due to its isolation from anthropogenic discharges but the findings of increased expression of the DRB and Complement cyt inhibitor genes indicated that this group may have elevated incidence of bacterial infection relative to two known healthy sea otters.

### Example 4

### Case Study: Adult Male Sea Otter from Katmai, Ak.

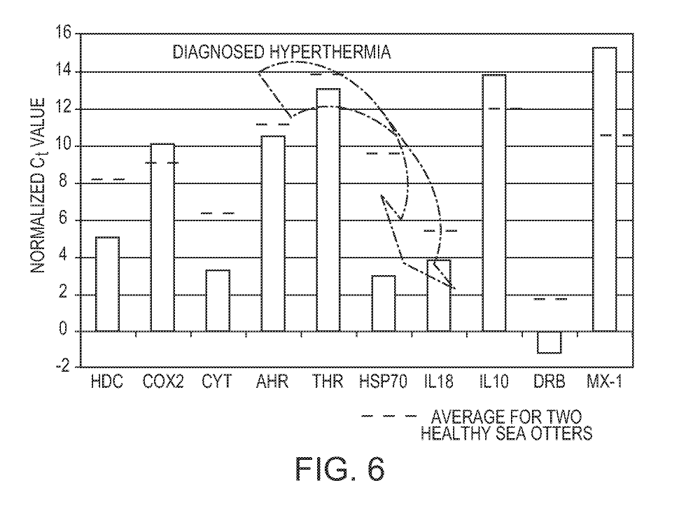

Blood sample was obtained from an adult male sea otter from the Katmai population that was diagnosed with hyperthermia by a veterinarian assisting with captures. Using the novel primers as described herein, we reaffirmed field diagnosis of hyperthermia compared to average values from two healthy sea otters (expression of HSP70 gene). We also identified the adult male sea otter's possible inability to combat viral disease (extreme lack of expression in MX-1 gene). See FIG. 6.

### Example 5

### Case Study: Adult Female Sea Otter from Katmai, Ak.

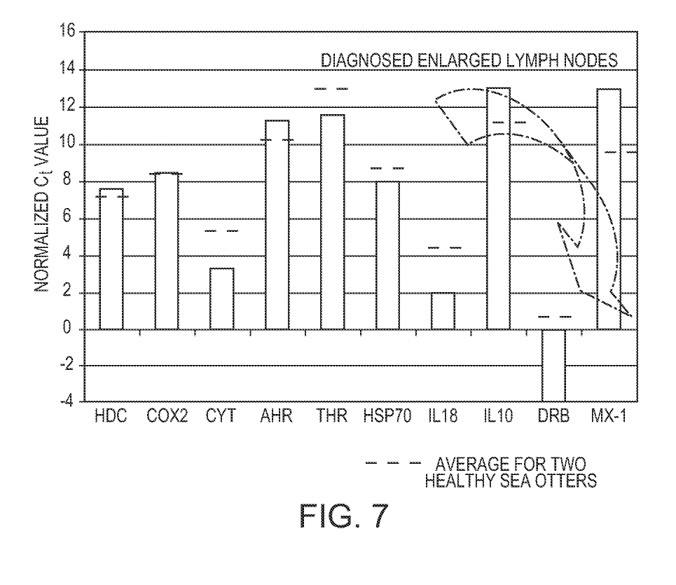

Blood sample was obtained from an adult female sea otter from Katmai population that was diagnosed with enlarged lymph nodes by a veterinarian assisting with captures. Using the novel primers as described herein, we diagnosed highly elevated expression of DRB suggesting bacterial infection that reaffirms the field diagnosis—see FIG. 7.

### Example 6

### Case Study: Monterey, Calif. and Big Sur, Calif. Sea Otters

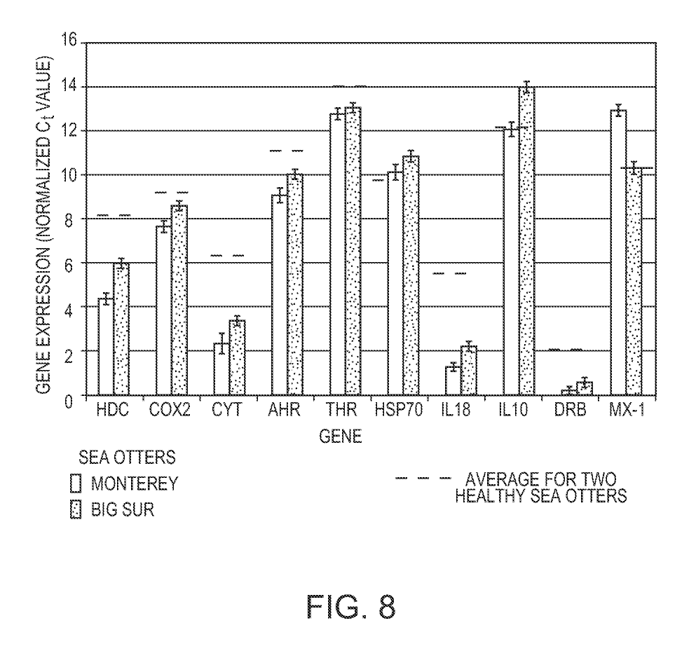

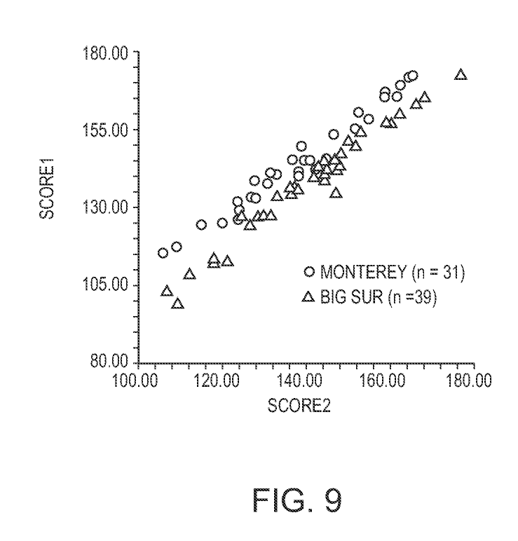

Blood samples were drawn from thirty-one (31) sea otters from Monterey, Calif. and thirty-nine (39) sea otters from Big Sur, Calif. Using the novel primers as described herein, the levels of expression in a suite of genes for each was detected—suite of genes set forth in FIG. 8. The average levels of expression in the suite of genes for all of the Monterey sea otters, and the average levels of expression in the suite of genes for all of the Big sur sea otters were determined. These average levels of expression in the suite of genes relative to the average expression values from two healthy sea otters is set forth in FIG. 8. A scatter plot showing the results of a discriminant function analysis comparing the levels of gene expression between two populations of sea otters (Monterey vs. Big Sur) is shown at FIG. 9.

### Example 7

### Case Study: Knight/Montague/Katmai Sea Otters

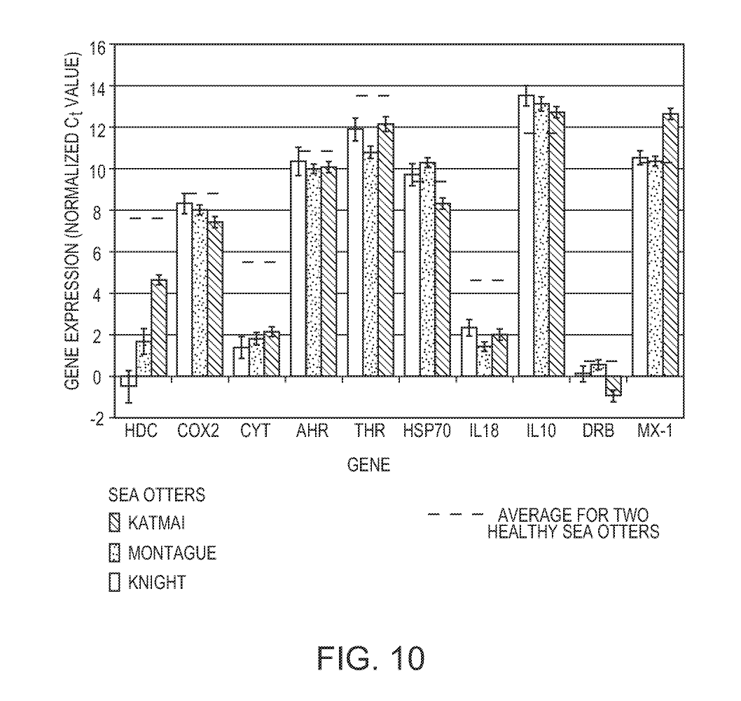

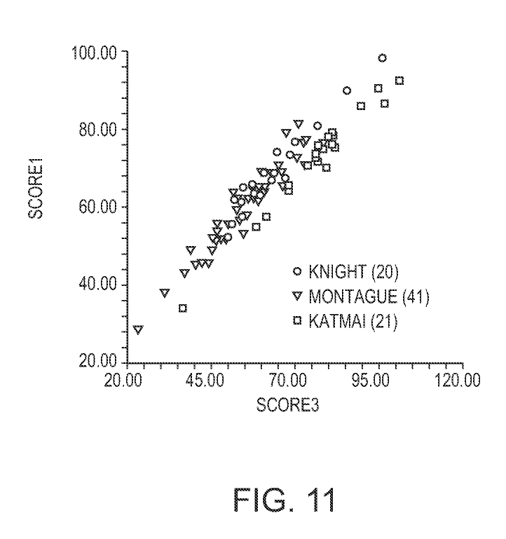

Blood samples were drawn from twenty (20) sea otters from Knight Island, forty-one (41) sea otters from Montague Island, and twenty-one (21) sea otters from Katmai, Ak. Using the novel primers as described herein, the levels of expression in a suite of genes for each was detected—suite of genes set forth in FIG. 10. The average levels of expression in the suite of genes for all of the sea otters from each given location were determined. These average levels of expression in the suite of genes relative to the average expression values from two healthy sea otters is set forth in FIG. 10. The Knight and Montague Island sea otters captured in 2006-2008 were from Prince William Sound, Alaska, the area impacted by the Exxon Valdez Oil spill in 1989. A scatter plot showing the results of a discriminant function analysis comparing the levels of gene expression between three populations of sea otters (Knight vs. Montague vs. Katmai) is shown at FIG. 11.

At the time of this filing, all field data are presented relative to two clinically diagnosed, healthy captive zoo animals that are provided for example and do not constitute a reference for free-ranging or wild sea animals. Further planned sampling from such animals will be necessary to establish ranges of values for healthy animals that can be used for comparisons in field studies. However, the findings demonstrate diagnostic utility in individual captive animals, as well as differences among populations of wild or free-ranging sea otters that are scientifically defensible, i.e., (1) relatively low expression in the assumed pristine Katmai, Ak. sea otter population; (2) increased gene expression, particularly the HDCMB21P gene implicated in tumorigenesis in sea otters from the area of Prince William Sound, Alaska, which suggests evidence of carcinogenesis possibly influenced by a major oil spill 20 years ago in the area where the sea otters were sampled; (3) increased expression in sea otters from the more urbanized Monterey Bay coastline than the more rural Big Sur coastline.

One having ordinary skill in the art with knowledge of the present invention would readily be able to establish ranges of values for healthy animals that can be used for comparisons in field studies and veterinary use. It is Applicants' intention that the present invention not be limited to ranges of values for healthy animals described and set forth herein.

The novel primer sets herein may be synthesized using conventional oligonucleotide synthesis methodologies.

The present invention demonstrates that a small sample of blood from a sea otter may be used to quantitatively assess expression of various genes, which levels of expression may be used as indicators of pathophysiological changes in sea otters, which may be used to assess and monitor sea otter health due to its exposure to fuels/oils or other toxins. Although each novel primer set herein may be run separately for the purpose described herein, one having ordinary skill in the art with knowledge of the present invention may wish to run more than one or, perhaps, all of the primer sets to best render a diagnostic assessment on the health of a sea otter.

Although the present invention is primarily described with reference to oil-exposed sea otters, the invention is not intended to be so limited. Applicants have identified a suite of genes that react to exposure to various toxins. Applicants submit that the present invention may be used to evaluate pathophysiological changes in sea otters that may have been exposed to toxins such as viruses, bacteria, or xenobiotics. Applicants submit that one skilled in the art with knowledge of the present invention would recognize that the present invention encompasses the use of the present invention to assess pathophysiological changes in sea otters that may have been exposed to any variety of environmental contaminants/toxicants.

Moreover, Applicants submit that the present invention may be used, in general, to monitor the health of sea otters. Applicants submit that the present invention has ecological and veterinary applications for monitoring the health of captive as well as free-ranging sea otters.

In addition, although the present invention is described with reference to sea otters, applicants submit that the invention is not intended to be so limited. Applicants submit that the present invention may be suitably employed to evaluate pathophysiological changes in Mustelidae family in general.

Applicants further submit that the present invention may be used by veterinarians in their treatment of otters and other species belonging to the Mustelidae family.

While particular embodiments of the present invention have been shown and described, it will be obvious to those skilled in the art that changes and modifications may be made without departing from this invention. Therefore, it is intended that the claims herein are to include all such obvious changes and modifications as fall within the true spirit and scope of this invention.

### REFERENCES

The teachings of the references cited herein are incorporated herein in their entirety:
(2008). QIAEX II® Handbook—For DNA extraction from Agarose and polyacrylamide gels and for desalting and concentrating DNA from solutions.
Altschul, S. F., W. Gish, et al. (1990). “Basic local alignment search tool.” J Mol Biol 215(3): 403-410.
Bartosiewicz, M., S. Penn, et al. (2001). “Applications of gene arrays in environmental toxicology: fingerprints of gene regulation associated with cadmium chloride, benzo(a)pyrene, and trichloroethylene.” Environ Health Perspect 109(1): 71-74.
Bowen, L. e. a. (2006). “Differential expression of immune response genes in Steller sea lions: an indicator of ecosystem health?” EcoHealth 3: 109-113.
Bowen, L. e. a. (2007). “Differential Gene Expression Induced by Exposure of Captive Mink to Fuel Oil: A Model for the Sea Otter.” EcoHealth 4: 298-309.
Burczynski, M. E., M. McMillian, et al. (2000). “Toxicogenomics-based discrimination of toxic mechanism in HepG2 human hepatoma cells.” Toxicol Sci 58(2): 399-415.
Invitrogen (2006). TOPO TA Cloning® Kit for Sequencing—Five-minute cloning of Taq polymerase-amplified PCR products for sequencing—User Manual, Version 0.
Marrack, P., T. Mitchell, et al. (2000). “Genomic-scale analysis of gene expression in resting and activated T cells.” Curr Opin Immunol 12(2): 206-209.
Mazet, J. A., I. A. Gardner, et al. (2001). “Effects of petroleum on mink applied as a model for reproductive success in sea otters.” J Wildl Dis 37(4): 686-692.
Mazet, J. K., I. A. Gardner, et al. (2000). “Evaluation of changes in hematologic and clinical biochemical values after exposure to petroleum products in mink (Mustela vison) as a model for assessment of sea otters (Enhydra lutris).” Am J Vet Res 61(10): 1197-1203.
PAXgene (2009). Blood RNA Kit Handbook—Version 2.
Sambook (2001). “Molecular Cloning: A Laboratory Manual, 3rd Edition.”
Schwartz, J. A., B. M. Aldridge, et al. (2004). “Chronic fuel oil toxicity in American mink (Mustela vison): systemic and hematological effects of ingestion of a low-concentration of bunker C fuel oil.” Toxicol Appl Pharmacol 200(2): 146-158.
Schwartz, J. A., B. M. Aldridge, et al. (2004). “Immunophenotypic and functional effects of bunker C fuel oil on the immune system of American mink (Mustela vison).” Vet Immunol Immunopathol 101(3-4): 179-190.

1. An oligonucleotide primer pair comprising a forward and a reverse primer, wherein said forward primer is SEQ ID NO. 1 (Enlu HDC F) and said reverse primer is SEQ ID NO. 2 (Enlu HDC R).

2. The oligonucleotide pair of claim 1, wherein said forward primer has at least 70% sequence identity with SEQ ID NO. 1 (Enlu HDC F).

3. The oligonucleotide pair of claim 1, wherein said forward primer has at least 80% sequence identity with SEQ ID NO. 1 (Enlu HDC F).

4. The oligonucleotide pair of claim 1, wherein said forward primer has at least 90% sequence identity with SEQ ID NO. 1 (Enlu HDC F).

5. The oligonucleotide pair of claim 1, wherein said reverse primer has at least 70% sequence identity with SEQ ID NO. 2 (Enlu HDC R).

6. The oligonucleotide pair of claim 1, wherein said reverse primer has at least 80% sequence identity with SEQ ID NO. 2 (Enlu HDC R).

7. The oligonucleotide pair of claim 1, wherein said reverse primer has at least 90% sequence identity with SEQ ID NO. 2 (Enlu HDC R).

8. The primer pair of claim 1, wherein said forward primer is SEQ ID NO. 3 (Enlu COX2 F) and said reverse primer is SEQ ID NO. 4 (Enlu COX2 R).

9. The oligonucleotide pair of claim 1, wherein said forward primer has at least 70% sequence identity with SEQ ID NO. 3 (Enlu COX2 F).

10. The oligonucleotide pair of claim 1, wherein said forward primer has at least 80% sequence identity with SEQ ID NO. 3 (Enlu COX2 F).

11. The oligonucleotide pair of claim 1, wherein said forward primer has at least 90% sequence identity with SEQ ID NO. 3 (Enlu COX2 F).

12. The oligonucleotide pair of claim 1, wherein said reverse primer has at least 70% sequence identity with SEQ ID NO. 4 (Enlu COX2 R).

13. The oligonucleotide pair of claim 1, wherein said reverse primer has at least 80% sequence identity with SEQ ID NO. 4 (Enlu COX2 R).

14. The oligonucleotide pair of claim 1, wherein said reverse primer has at least 90% sequence identity with SEQ ID NO. 4 (Enlu COX2 R).

15. The primer pair of claim 1, wherein said forward primer is SEQ ID NO. 5 (Enlu CYT F) and said reverse primer is SEQ ID NO. 6 (Enlu CYT R).

16. The oligonucleotide pair of claim 1, wherein said forward primer has at least 70% sequence identity with SEQ ID NO. 5 (Enlu CYT F).

17. The oligonucleotide pair of claim 1, wherein said forward primer has at least 80% sequence identity with SEQ ID NO. 5 (Enlu CYT F).

18. The oligonucleotide pair of claim 1, wherein said forward primer has at least 90% sequence identity with SEQ ID NO. 5 (Enlu CYT F).

19. The oligonucleotide pair of claim 1, wherein said reverse primer has at least 70% sequence identity with SEQ ID NO. 6 (Enlu CYT R).

20. The oligonucleotide pair of claim 1, wherein said reverse primer has at least 80% sequence identity with SEQ ID NO. 6 (Enlu CYT R).

21. The oligonucleotide pair of claim 1, wherein said reverse primer has at least 90% sequence identity with SEQ ID NO. 6 (Enlu CYT R).

22. The primer pair of claim 1, wherein said forward primer is SEQ ID NO. 7 (Enlu AHR F) and said reverse primer is SEQ ID NO. 8 (Enlu AHR R).

23. The oligonucleotide pair of claim 1, wherein said forward primer has at least 70% sequence identity with SEQ ID NO. 7 (Enlu AHR F).

24. The oligonucleotide pair of claim 1, wherein said forward primer has at least 80% sequence identity with SEQ ID NO. 7 (Enlu AHR F).

25. The oligonucleotide pair of claim 1, wherein said forward primer has at least 90% sequence identity with SEQ ID NO. 7 (Enlu AHR F).

26. The oligonucleotide pair of claim 1, wherein said reverse primer has at least 70% sequence identity with SEQ ID NO. 8 (Enlu AHR R).

27. The oligonucleotide pair of claim 1, wherein said reverse primer has at least 80% sequence identity with SEQ ID NO. 8 (Enlu AHR R).

28. The oligonucleotide pair of claim 1, wherein said reverse primer has at least 90% sequence identity with SEQ ID NO. 8 (Enlu AHR R).

29. The primer pair of claim 1, wherein said forward primer is SEQ ID NO. 9 (Enlu CYP F) and said reverse primer is SEQ ID NO. 10 (Enlu CYP R).

30. The oligonucleotide pair of claim 1, wherein said forward primer has at least 70% sequence identity with SEQ ID NO. 9 (Enlu CYP F).

31. The oligonucleotide pair of claim 1, wherein said forward primer has at least 80% sequence identity with SEQ ID NO. 9 (Enlu CYP F).

32. The oligonucleotide pair of claim 1, wherein said forward primer has at least 90% sequence identity with SEQ ID NO. 9 (Enlu CYP F).

33. The oligonucleotide pair of claim 1, wherein said reverse primer has at least 70% sequence identity with SEQ ID NO. 10 (Enlu CYP R).

34. The oligonucleotide pair of claim 1, wherein said reverse primer has at least 80% sequence identity with SEQ ID NO. 10 (Enlu CYP R).

35. The oligonucleotide pair of claim 1, wherein said reverse primer has at least 90% sequence identity with SEQ ID NO. 10 (Enlu CYP R).

36. The primer pair of claim 1, wherein said forward primer is SEQ ID NO. 11 (Enlu THR F) and said reverse primer is SEQ ID NO. 12 (Enlu THR R).

37. The oligonucleotide pair of claim 1, wherein said forward primer has at least 70% sequence identity with SEQ ID NO. 11 (Enlu THR F).

38. The oligonucleotide pair of claim 1, wherein said forward primer has at least 80% sequence identity with SEQ ID NO. 11 (Enlu THR F).

39. The oligonucleotide pair of claim 1, wherein said forward primer has at least 90% sequence identity with SEQ ID NO. 11 (Enlu THR F).

40. The oligonucleotide pair of claim 1, wherein said reverse primer has at least 70% sequence identity with SEQ ID NO. 12 (Enlu THR R).

41. The oligonucleotide pair of claim 1, wherein said reverse primer has at least 80% sequence identity with SEQ ID NO. 12 (Enlu THR R).

42. The oligonucleotide pair of claim 1, wherein said reverse primer has at least 90% sequence identity with SEQ ID NO. 12 (Enlu THR R).

43. The primer pair of claim 1, wherein said forward primer is SEQ ID NO. 13 (Enlu HSP70 F) and said reverse primer is SEQ ID NO. 14 (Enlu HSP70 R).

44. The oligonucleotide pair of claim 1, wherein said forward primer has at least 70% sequence identity with SEQ ID NO. 13 (Enlu HSP70 F).

45. The oligonucleotide pair of claim 1, wherein said forward primer has at least 80% sequence identity with SEQ ID NO. 13 (Enlu HSP70 F).

46. The oligonucleotide pair of claim 1, wherein said forward primer has at least 90% sequence identity with SEQ ID NO. 13 (Enlu HSP70 F).

47. The oligonucleotide pair of claim 1, wherein said reverse primer has at least 70% sequence identity with SEQ ID NO. 14 (Enlu HSP70 R).

48. The oligonucleotide pair of claim 1, wherein said reverse primer has at least 80% sequence identity with SEQ ID NO. 14 (Enlu HSP70 R).

49. The oligonucleotide pair of claim 1, wherein said reverse primer has at least 90% sequence identity with SEQ ID NO. 14 (Enlu HSP70 R).

50. The primer pair of claim 1, wherein said forward primer is SEQ ID NO. 15 (Enlu IL18 F) and said reverse primer is SEQ ID NO. 16 (Enlu IL18 R).

51. The oligonucleotide pair of claim 1, wherein said forward primer has at least 70% sequence identity with SEQ ID NO. 15 (Enlu IL18 F).

52. The oligonucleotide pair of claim 1, wherein said forward primer has at least 80% sequence identity with SEQ ID NO. 15 (Enlu IL18 F).

53. The oligonucleotide pair of claim 1, wherein said forward primer has at least 90% sequence identity with SEQ ID NO. 15 (Enlu IL18 F).

54. The oligonucleotide pair of claim 1, wherein said reverse primer has at least 70% sequence identity with SEQ ID NO. 16 (Enlu IL18 R).

55. The oligonucleotide pair of claim 1, wherein said reverse primer has at least 80% sequence identity with SEQ ID NO. 16 (Enlu IL18 R).

56. The oligonucleotide pair of claim 1, wherein said reverse primer has at least 90% sequence identity with SEQ ID NO. 16 (Enlu IL18 R).

57. The primer pair of claim 1, wherein said forward primer is SEQ ID NO. 17 (Enlu IL10 F) and said reverse primer is SEQ ID NO. 18 (Enlu IL10 R).

58. The oligonucleotide pair of claim 1, wherein said forward primer has at least 70% sequence identity with SEQ ID NO. 17 (Enlu IL10 F).

59. The oligonucleotide pair of claim 1, wherein said forward primer has at least 80% sequence identity with SEQ ID NO. 17 (Enlu IL10 F).

60. The oligonucleotide pair of claim 1, wherein said forward primer has at least 90% sequence identity with SEQ ID NO. 17 (Enlu IL10 F).

61. The oligonucleotide pair of claim 1, wherein said reverse primer has at least 70% sequence identity with SEQ ID NO. 18 (Enlu IL10 R).

62. The oligonucleotide pair of claim 1, wherein said reverse primer has at least 80% sequence identity with SEQ ID NO. 18 (Enlu IL10 R).

63. The oligonucleotide pair of claim 1, wherein said reverse primer has at least 90% sequence identity with SEQ ID NO. 18 (Enlu IL10 R).

64. The primer pair of claim 1, wherein said forward primer is SEQ ID NO. 19 (Enlu DRB F) and said reverse primer is SEQ ID NO. 20 (Enlu DRB R).

65. The oligonucleotide pair of claim 1, wherein said forward primer has at least 70% sequence identity with SEQ ID NO. 19 (Enlu DRB F).

66. The oligonucleotide pair of claim 1, wherein said forward primer has at least 80% sequence identity with SEQ ID NO. 19 (Enlu DRB F).

67. The oligonucleotide pair of claim 1, wherein said forward primer has at least 90% sequence identity with SEQ ID NO. 19 (Enlu DRB F).

68. The oligonucleotide pair of claim 1, wherein said reverse primer has at least 70% sequence identity with SEQ ID NO. 20 (Enlu DRB R).

69. The oligonucleotide pair of claim 1, wherein said reverse primer has at least 80% sequence identity with SEQ ID NO. 20 (Enlu DRB R).

70. The oligonucleotide pair of claim 1, wherein said reverse primer has at least 90% sequence identity with SEQ ID NO. 20 (Enlu DRB R).

71. The primer pair of claim 1, wherein said forward primer is SEQ ID NO. 21 (Enlu Met F) and said reverse primer is SEQ ID NO. 22 (Enlu Met R).

72. The oligonucleotide pair of claim 1, wherein said forward primer has at least 70% sequence identity with SEQ ID NO. 21 (Enlu Met F).

73. The oligonucleotide pair of claim 1, wherein said forward primer has at least 80% sequence identity with SEQ ID NO. 21 (Enlu Met F).

74. The oligonucleotide pair of claim 1, wherein said forward primer has at least 90% sequence identity with SEQ ID NO. 21 (Enlu Met F).

75. The oligonucleotide pair of claim 1, wherein said reverse primer has at least 70% sequence identity with SEQ ID NO. 22 (Enlu Met R).

76. The oligonucleotide pair of claim 1, wherein said reverse primer has at least 80% sequence identity with SEQ ID NO. 22 (Enlu Met R).

77. The oligonucleotide pair of claim 1, wherein said reverse primer has at least 90% sequence identity with SEQ ID NO. 22 (Enlu Met R).

78. The primer pair of claim 1, wherein said forward primer is SEQ ID NO. 23 (Enlu CIRBP F) and said reverse primer is SEQ ID NO. 24 (Enlu CIRBP R).

79. The oligonucleotide pair of claim 1, wherein said forward primer has at least 70% sequence identity with SEQ ID NO. 23 (Enlu CIRBP F).

80. The oligonucleotide pair of claim 1, wherein said forward primer has at least 80% sequence identity with SEQ ID NO. 23 (Enlu CIRBP F).

81. The oligonucleotide pair of claim 1, wherein said forward primer has at least 90% sequence identity with SEQ ID NO. 23 (Enlu CIRBP F).

82. The oligonucleotide pair of claim 1, wherein said reverse primer has at least 70% sequence identity with SEQ ID NO. 24 (Enlu CIRBP R).

83. The oligonucleotide pair of claim 1, wherein said reverse primer has at least 80% sequence identity with SEQ ID NO. 24 (Enlu CIRBP R).

84. The oligonucleotide pair of claim 1, wherein said reverse primer has at least 90% sequence identity with SEQ ID NO. 24 (Enlu CIRBP R).

85. The primer pair of claim 1, wherein said forward primer is SEQ ID NO. 25 (Enlu MX1 express F) and said reverse primer is SEQ ID NO. 26 (Enlu MX1express R).

86. The oligonucleotide pair of claim 1, wherein said forward primer has at least 70% sequence identity with SEQ ID NO. 25 (Enlu MX1express F).

87. The oligonucleotide pair of claim 1, wherein said forward primer has at least 80% sequence identity with SEQ ID NO. 25 (Enlu MX1express F).

88. The oligonucleotide pair of claim 1, wherein said forward primer has at least 90% sequence identity with SEQ ID NO. 25 (Enlu MX1express F).

89. The oligonucleotide pair of claim 1, wherein said reverse primer has at least 70% sequence identity with SEQ ID NO. 26 (Enlu MX1express R).

90. The oligonucleotide pair of claim 1, wherein said reverse primer has at least 80% sequence identity with SEQ ID NO. 26 (Enlu MX1express R).

91. The oligonucleotide pair of claim 1, wherein said reverse primer has at least 80% sequence identity with SEQ ID NO. 26 (Enlu MX1express R).

92. The oligonucleotide pair of claim 1, wherein said reverse primer has at least 90% sequence identity with SEQ ID NO. 26 (Enlu MX1express R).

93. The primer pair of claim 1, wherein said forward primer is SEQ ID NO. 27 (Enlu IL2 F) and said reverse primer is SEQ ID NO. 28 (Enlu IL2 R).

94. The oligonucleotide pair of claim 1, wherein said forward primer has at least 70% sequence identity with SEQ ID NO. 27 (Enlu IL2 F).

95. The oligonucleotide pair of claim 1, wherein said forward primer has at least 80% sequence identity with SEQ ID NO. 27 (Enlu IL2 F).

96. The oligonucleotide pair of claim 1, wherein said forward primer has at least 90% sequence identity with SEQ ID NO. 27 (Enlu IL2 F).

97. The oligonucleotide pair of claim 1, wherein said reverse primer has at least 70% sequence identity with SEQ ID NO. 28 (Enlu IL2 R).

98. The oligonucleotide pair of claim 1, wherein said reverse primer has at least 80% sequence identity with SEQ ID NO. 28 (Enlu IL2 R).

99. The oligonucleotide pair of claim 1, wherein said reverse primer has at least 90% sequence identity with SEQ ID NO. 28 (Enlu IL2 R).

100. The primer pair of claim 1, wherein said forward primer is SEQ ID NO. 29 (Enlu S9 F) and said reverse primer is SEQ ID NO. 30 (Enlu S9 R).

101. The oligonucleotide pair of claim 1, wherein said forward primer has at least 70% sequence identity with SEQ ID NO. 29 (Enlu S9 F).

102. The oligonucleotide pair of claim 1, wherein said forward primer has at least 80% sequence identity with SEQ ID NO. 29 (Enlu S9 F).

103. The oligonucleotide pair of claim 1, wherein said forward primer has at least 90% sequence identity with SEQ ID NO. 29 (Enlu S9 F).

104. The oligonucleotide pair of claim 1, wherein said reverse primer has at least 70% sequence identity with SEQ ID NO. 30 (Enlu S9 R).

105. The oligonucleotide pair of claim 1, wherein said reverse primer has at least 80% sequence identity with SEQ ID NO. 30 (Enlu S9 R).

106. The oligonucleotide pair of claim 1, wherein said reverse primer has at least 90% sequence identity with SEQ ID NO. 30 (Enlu S9 R).

107. A method for assessing the health of at least one sea otter, wherein said method comprises:
(a) obtaining a sample from a sea otter;
(b) amplifying nucleic acid from said sample using at least one oligonucleotide primer pair comprising a forward primer and a reverse primer, each primer having at least 70% sequence identity with the corresponding forward and reverse primers selected from the group consisting of the following primer sequences: (SEQ ID NOS:1:2), (SEQ ID NOS:3:4), (SEQ ID NOS:5:6), (SEQ ID NOS:7:8), (SEQ ID NOS:9:10), (SEQ ID NOS:11:12), (SEQ ID NOS:13:14), (SEQ ID NOS:15:16), (SEQ ID NOS:17:18), (SEQ ID NOS:19:20), (SEQ ID NOS:21:22), (SEQ ID NOS:23:24), (SEQ ID NOS:25:26), (SEQ ID NOS:27:28), (SEQ ID NOS:29:30);
(c) determining the expression levels of the genes corresponding to said primer pair, and;
(d) comparing the expression levels of said genes in said sample to known control mean expression values of each of the genes.

108. The method of claim 107, wherein said pathophysiological changes measured are changes due to exposure to crude oil.

109. A kit for assessing the health of at least one sea otter, wherein said kit comprises: at least one oligonucleotide primer pair comprising a forward primer and a reverse primer, each primer having at least 70% sequence identity with the corresponding forward and reverse primers selected from the group consisting of the following primer sequences: (SEQ ID NOS:1:2), (SEQ ID NOS:3:4), (SEQ ID NOS:5:6), (SEQ ID NOS:7:8), (SEQ ID NOS:9:10), (SEQ ID NOS:11:12), (SEQ ID NOS:13:14), (SEQ ID NOS:15:16), (SEQ ID NOS:17:18), (SEQ ID NOS:19:20), (SEQ ID NOS:21:22), (SEQ ID NOS:23:24), (SEQ ID NOS:25:26), (SEQ ID NOS:27:28), (SEQ ID NOS:29:30).

110. The kit of claim 109 further comprising instructions for their use in assessing the health of said otter.

111. The kit of claim 110 further comprising panel probes sufficient to determine the identity of the sea otter gene.

112. (canceled)

113. (canceled)
